(19) United States
(12) Patent Application Publication
(10) Pub. No.: US 2025/0046405 A1
(43) Pub. Date: Feb. 6, 2025

(54) DATA PROCESSING SYSTEM INCLUDING REMOTE PROCESSING DEVICE, AND OPERATION METHOD THEREOF

(71) Applicants: SK Hynix Inc., Icheon (KR); Daegu Gyeongbuk Institute of Science and Technology, Daegu (KR)

(72) Inventors: Sanghoon LEE, Daegu (KR); Jongho Park, Daegu (KR); Yeseong Kim, Daegu (KR); Minho Ha, Icheon (KR); Byungil Koh, Icheon (KR); Jungmin Choi, Icheon-si (KR)

(21) Appl. No.: 18/420,542

(22) Filed: Jan. 23, 2024

(30) Foreign Application Priority Data
Aug. 4, 2023 (KR) 10-2023-0102378
Dec. 18, 2023 (KR) 10-2023-0184867

(51) Int. Cl. G16C 20/90

(52) U.S. Cl. CPC G16C 20/90

(57) ABSTRACT

A data processing system includes a host configured to execute a program for processing a given data set; and a remote processing device coupled to the host via an interface, wherein the host includes a profile control circuit configured to generate a profile corresponding to a function that is called during execution of the program; a profile database storing execution location of the function corresponding to the profile; and a policy execution circuit configured to allocate the function called during the execution of the program to the host or the remote processing device based on the execution location the profile database.

### CROSS-REFERENCE TO RELATED APPLICATION

The present application claims priority under 35 U.S.C. § 119(a) to Korean Patent Application No. 10-2023-0102378, filed on Aug. 4, 2023, and Korean Patent Application No. 10-2023-0184867, filed on Dec. 18, 2023, which are incorporated herein by reference in their entireties.

### BACKGROUND

### 1. Technical Field

Embodiments generally relate to a data processing system and an operation method thereof, and more particularly, to a data processing system that executes a program, processing large amounts of data distributed between a host and a remote processing device, and an operation method thereof.

### 2. Related Art

When a data processing system executes a program that processes large amounts of data, like a clustering program, there is a need for an exceptionally large main memory capacity. In addition, the communication costs between a central processing unit (CPU) and a memory tend to increase significantly.

Accordingly, a technology for distributed processing by combining near data processing (NDP) devices that include a remote memory connected to a host is being proposed.

However, compared to accessing the main memory, accessing the remote memory has relatively higher latency, and bandwidth is limited due to interface constraints, which hampers the efficiency of distributed processing.

### SUMMARY

In accordance with an embodiment of the present disclosure, a data processing system may include a host configured to execute a program for processing a given data set; and a remote processing device coupled to the host via an interface, wherein the host includes a profile control circuit configured to generate a profile corresponding to a function that is called during execution of the program; a profile database storing execution location of the function corresponding to the profile; and a policy execution circuit configured to allocate the function called during the execution of the program to the host or the remote processing device based on the execution location stored in the profile database.

In accordance with an embodiment of the present disclosure, an operation method of a data processing system including a host executing a program that processes a given data set and a remote processing device coupled to the host via an interface, the operation method may comprise performing a profile operation to store a plurality of profiles corresponding to a plurality of functions called during execution of the program with a synthesized data set having a size smaller than that of the given data set; and performing an optimization operation to decide an optimal execution location for each of the plurality of functions either on the host or the remote processing device while ethe program is executed with the synthesized data set.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate various embodiments, and explain various principles and advantages of those embodiments.

FIG. 1 illustrates a data processing system according to an embodiment of the present disclosure.

FIG. 2 illustrates a profile operation of a data processing system according to an embodiment of the present disclosure.

FIG. 3 illustrates a data structure of a profile database according to an embodiment of the present disclosure.

FIG. 4 is a flowchart showing an optimization operation of a data processing system according to an embodiment of the present disclosure.

FIG. 5 illustrates an optimization operation of a data processing system according to an embodiment of the present disclosure.

### DETAILED DESCRIPTION

The following detailed description references the accompanying figures in describing illustrative embodiments consistent with this disclosure. The embodiments are provided for illustrative purposes and are not exhaustive. Additional embodiments not explicitly illustrated or described are possible. Further, modifications can be made to presented embodiments within the scope of teachings of the present disclosure. The detailed description is not meant to limit this disclosure. Rather, the scope of the present disclosure is defined in accordance with claims and equivalents thereof. Also, throughout the specification, reference to “an embodiment” or the like is not necessarily to only one embodiment, and different references to any such phrase are not necessarily to the same embodiment(s).

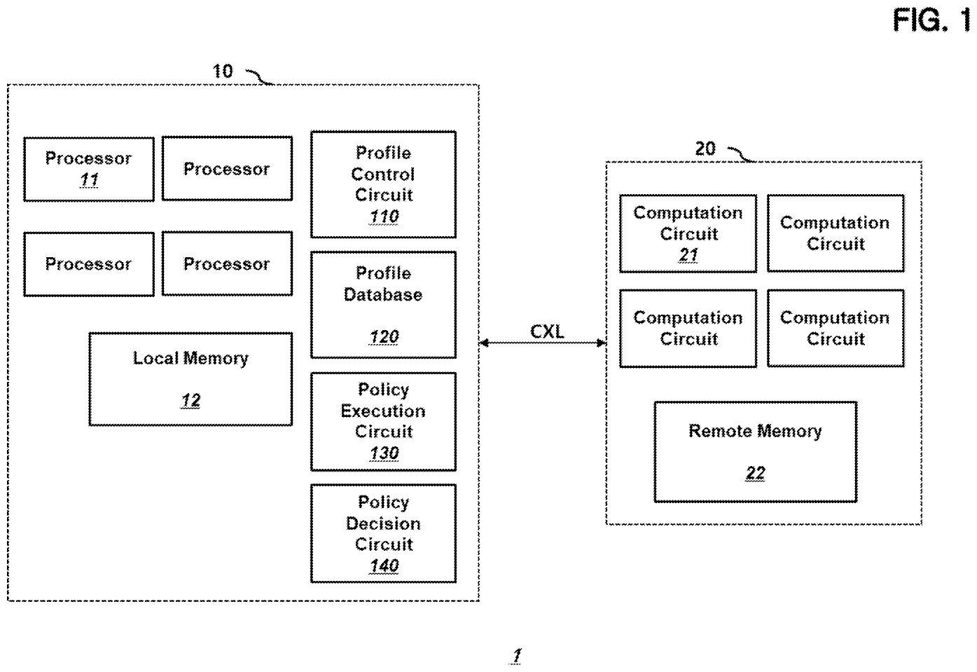

FIG. 1 illustrates a data processing system 1 according to an embodiment of the present disclosure.

The data processing system 1 includes a host 10 and a remote processing device 20.

The host 10 includes one or more processors 11 and a local memory 12. The processor 11 may be a central processing unit (CPU), and the local memory 12 may be referred to as a main memory.

The host 10 further includes a profile control circuit 110, a profile database 120, a policy execution circuit 130, and a policy decision circuit 140.

Operations of the profile control circuit 110, the profile database 120, the policy execution circuit 130, and the policy decision circuit 140 are described in detail below.

The remote processing device 20 supports near data processing (NDP), and thus may be referred to as an NDP device.

The remote processing device 20 includes one or more computation circuits 21 and a remote memory 22. The computation circuit 21 performs an NDP function using data stored in the remote memory 22.

The technology itself regarding the remote processing device 20, which includes the computation circuit 21 and the remote memory 22 and performs the NDP function, is well-known.

The host 10 and the remote processing device 20 may be connected to each other using various interface technologies.

In this embodiment, it is assumed that the host 10 and the remote processing device 20 are connected to each other using an interface that supports type-2 standard of compute express link (CXL) technology.

Since the type-2 standard of CXL technology itself is well known, detailed disclosure of the interface supporting the CXL technology is omitted.

When supporting the CXL technology, the processor 11 of the host 10 can access the remote memory 22 in the same way it reads/writes to the local memory 12.

Through this interface technology, the host 10 can offload functions and data to the remote memory 22 and subsequently receive processing results of the functions.

The data processing system 1 can process some functions on the host 10 and also process some functions on the remote processing device 20, especially for a program that processes large amounts of data.

Hereinafter, the present invention is disclosed using a clustering program as an example.

The clustering program is a technology that categorizes data within a dataset using an unsupervised method.

Since there are various clustering programs depending on clustering algorithms used therein, a distributed processing method optimized for a specific program may not be equally appliable to other programs.

In this embodiment, the host 10 sequentially performs a profile operation for various functions included in the program and an optimization operation for allocating the functions to targets, thereby realizing automated allocation of functions called while the program is executed.

At this time, the target corresponds to either the host 10 or the remote processing device 20.

In this embodiment, a program context associated with a call path of a function is used to create a profile corresponding to the function, and a genetic algorithm is applied for optimization, but the scope of the present invention is not limited thereto.

In the profile operation and the optimization operation, the clustering program runs using a synthesized dataset rather than an actual dataset. The synthesized dataset can be created randomly or by sampling a portion of the actual dataset.

First, the profile operation is disclosed below.

While the program is executed, various functions are called.

In this embodiment, the profile operation is performed using a program context corresponding to a function rather than the function itself.

Through this, a profile corresponding to the function is created by reflecting various contextual information in which the function is called.

In this embodiment, the program context reflects a series of function calls stacked in a stack memory.

More specifically, in this embodiment, a hashed value of an array of return addresses corresponding to a series of functions stacked in the stack memory is used as a program context corresponding to a currently called function, and the hashed value is, for example, a 32-bit value.

Accordingly, even the same function can have different program contexts depending on the context in which it is called.

Hereinafter, a profile and a program context can be used interchangeably.

The profile control circuit 110 determines a profile corresponding to a function. In this embodiment, as described above, a profile corresponding to a function is generated by hashing an array of return addresses of the functions stacked in the stack memory when the function is called.

The profile database 120 stores profiles created to correspond to called functions.

In this embodiment, the profile database 120 stores a profile corresponding to a called function and associates it with a type of the called function.

In this embodiment, a type of a function is classified into a computation function or a memory function. For example, the memory function is a function that controls a memory, such as a function allocating a memory, and the computation function is any function other than the memory function.

For example, computation functions may include thread allocation functions and general functions that perform general operations other than the thread allocation.

When storing a new profile, a relationship between profiles can be stored by identifying the context of the program.

For example, when a memory function is called during the process of performing a computation function, the computation function and the memory function can be viewed as related.

Accordingly, after a profile corresponding to each function is created, the relationship between the two profiles can be stored together with the two profiles, and thus searching one profile can reveal the associated other profile.

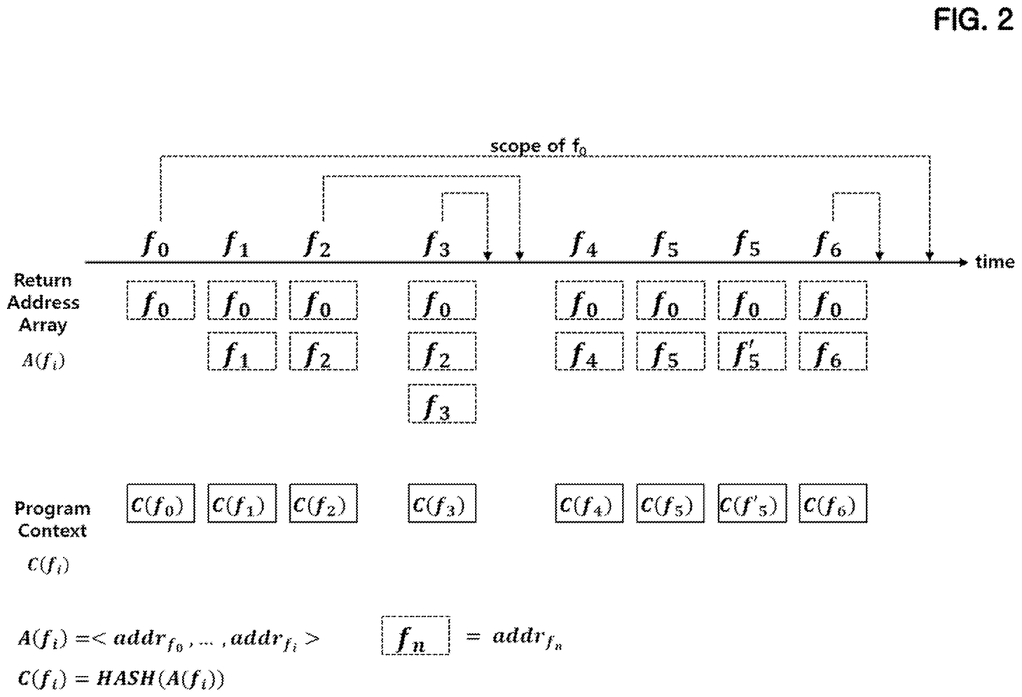

FIG. 2 illustrates the profile operation of the data processing system 1 according to an embodiment of the present disclosure.

FIG. 2 shows that a plurality of functions f₀ to f₆ are called sequentially, and the survival scope of each function is indicated by an arrow. For a function that does not have a corresponding arrow, it indicates that the function returns before the next function is called.

A dotted rectangle represents a return address corresponding to a function. Even for the same function, a return address may vary depending on when it is called. For example, for the function f₅, f₅ and ′ each displayed with a dotted rectangle indicate that they were called at different times and thus have different return addresses.

In FIG. 2, f₁ and f₅ correspond to memory allocation functions, f₄ corresponds to a thread creation function, and the remaining functions correspond to general functions. That is, f₁ and f₅ each correspond to the memory function and f₄ and the remaining functions each correspond to the computation function.

FIG. 2 shows when functions are called and the survival scope of the called functions. For example, f₀ is called and terminates its operation after the operation of f₆ is completed.

In this embodiment, a return address array represents an array containing the return addresses of the currently executing procedures or functions stored in the stack memory.

Referring to FIG. 2, a return address array A(fᵢ) corresponding to a function fᵢ corresponds to an array of return addresses sequentially stored in the stack memory when the function fᵢ is called.

When f₀ is called first, a return address of f₀ is stored in the stack memory. After that, according to a function that is subsequently called, a return address of the subsequently called function is stored in the stack memory.

For example, when f₁ is called, a return address of f₁ is stored after the return address of f₀, when f₂ is called after f₁ returns, a return address of f₂ is stored after the return address of f₀, and then when f₃ is called, return addresses of f₀, f₂, and f₃ are sequentially stored in the stack memory.

In this embodiment, a profile corresponding to the function fᵢ corresponds to a hashed value of the return address array A(fᵢ) corresponding to the function fᵢ.

At this time, a hash function receives a return address array as an input. For example, if the hash function is denoted as HASH( ), the profile corresponding to f₀ can be expressed as C(f₀)=HASH(<addr_{f0}>), and the profile corresponding to f₁ can be expressed as C(f₁)=HASH (<addr_{f0}, addr_{f1}>).

When a clustering program is executed, functions are called sequentially. The profile control circuit 110 then stores profiles, created based on the above-described principle, in the profile database 120.

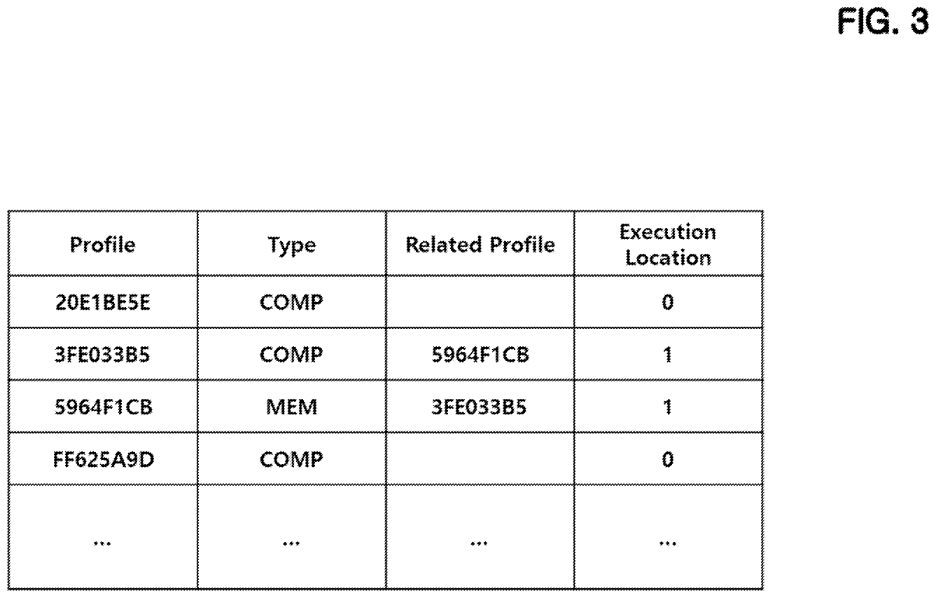

FIG. 3 illustrates a data structure of the profile database 120 according to an embodiment of the present disclosure.

The profile database 120 includes a ‘profile’ field, a ‘type’ field, a ‘related profile’ field, and an ‘execution location’ field.

The profile field stores a profile created as described above.

The type field stores a type of a profile that is classified into a computation operation COMP or a memory operation MEM according to a type of a corresponding function.

For example, types of profiles corresponding to the general functions and the thread creation functions are designated as COMP, and a type of a profile corresponding to the memory allocation function is designated as MEM.

The related profile field stores a profile associated with a current profile.

For example, if the type of the current profile is MEM, a profile corresponding to the computation function, which calls the memory allocation function, is saved in the related profile field.

For example, in FIG. 2, f₁, which is the memory allocation function, is called after f₀, which is the computation function.

When applying this to FIG. 3, assuming that the profile of f₀ is “3FE033B5” and the profile of f₁ is “5964F1CB,” the profile of f₀, “3FE033B5,” can be stored as an associated profile of the profile of f₁, “5964F1CB”. Additionally, the profile of f₁, “5964F1CB,” can be additionally stored as an associated profile of the profile of f₀, “3FE033B5”.

The execution location field stores the location where a function corresponding to a profile will be executed. For example, when a function corresponding to a profile is executed on the host 10, “0” can be stored, and when the corresponding function is executed on the remote processing device 20, “1” can be stored.

Types of profiles and related profiles can be used in the optimization operation, and thus values of the execution location field are finally determined through the optimization operation.

In this way, the profile database 120 is created using all functions called while executing the clustering program, and then the optimization operation is performed thereon.

Below, the optimization operation is described.

The policy decision circuit 140 determines where each profile stored in the profile database 120 should be executed, either on the host 10 or the remote processing device 20. The policy execution circuit 130 instructs a function to be executed either on the host 10 or on the remote processing device 20, based on the determined execution location of a corresponding profile.

The policy decision circuit 140 controls the optimization operation and determines the execution location corresponding to each profile according to an optimization algorithm.

Although various algorithms can be used as the optimization algorithm, the present embodiment is described below using the genetic algorithm as an example.

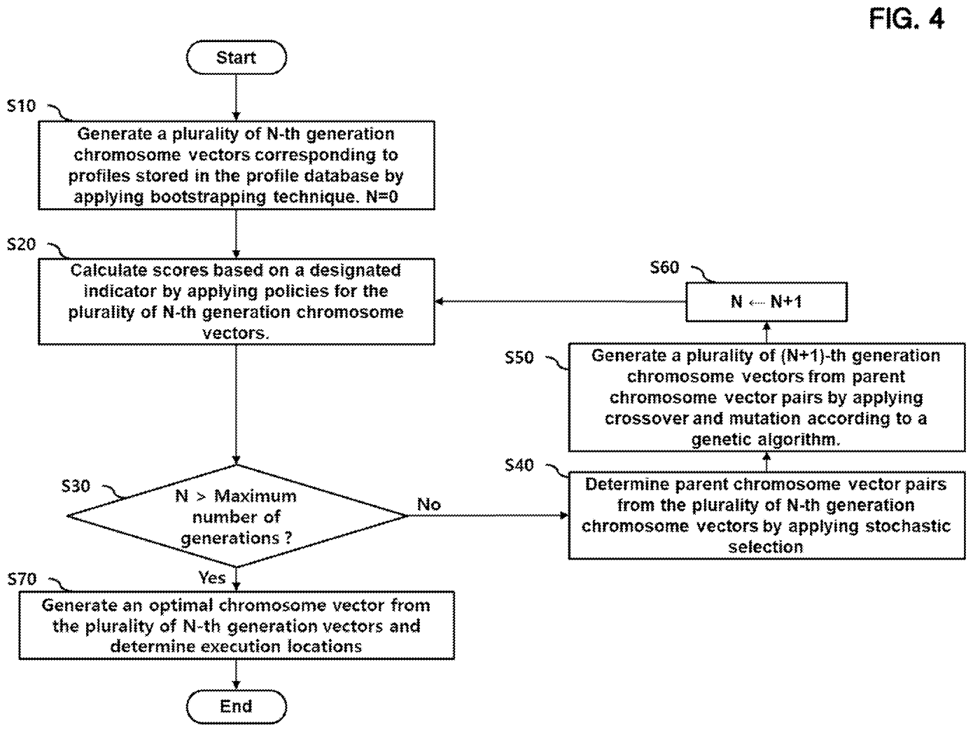

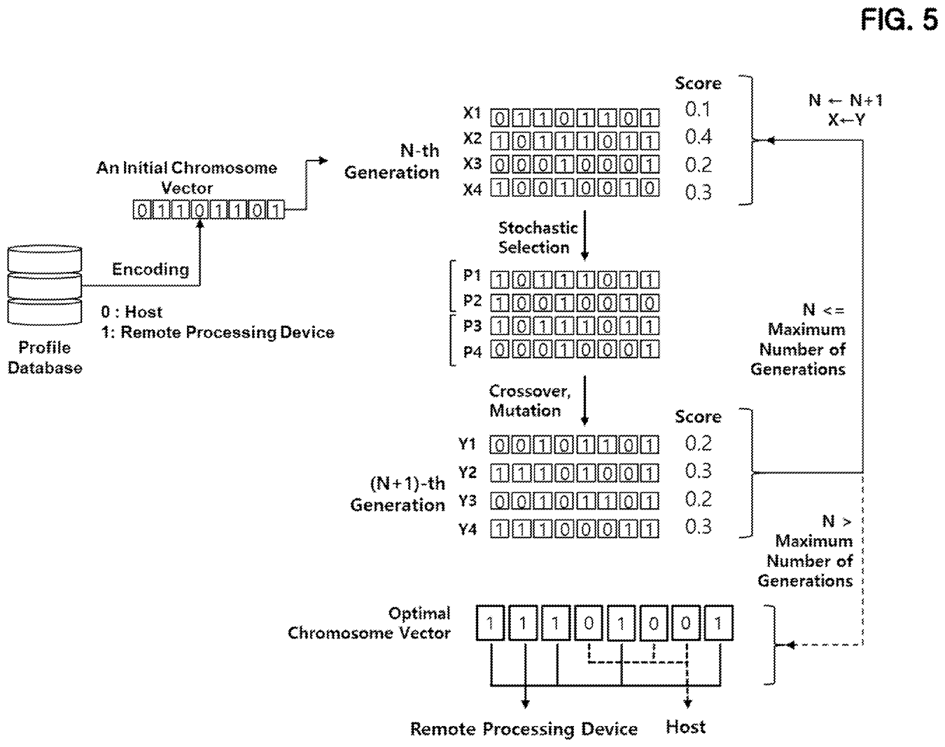

FIG. 4 is a flowchart showing the optimization operation of the data processing system 1 according to an embodiment of the present disclosure, and FIG. 5 is a diagram for describing the optimization operation.

First, at S10, a plurality of N-th generation chromosome vectors corresponding to profiles included in the profile database 120 are generated. Each chromosome vector includes a plurality of elements that respectively correspond to the profiles included in the profile database 120.

A generation variable N, which is a non-negative integer, is initialized as 0. Hereinafter, a 0-th generation chromosome vector may be represented as an initial chromosome vector.

In the present disclosure, number of chromosome vectors corresponding to a generation is the same throughout the generations, but the present invention is not limited thereto. In FIG. 5, number of chromosome vectors corresponding to each generation is 4.

Therefore, each element of an initial chromosome vector corresponds to a corresponding one of the profiles included in the profile database 120, and its value, i.e., an element value, indicates the execution location of a function corresponding to the profile.

For example, the element value is set to “0” when the execution location is the host 10, and “1” when the execution location is the remote processing device 20.

While elements of an initial chromosome vector can be assigned completely random values, the operation of the genetic algorithm may either fail to produce a converged chromosome vector or require an excessive amount of time to converge.

In this embodiment, bootstrapping technology is employed to counteract this phenomenon. In this embodiment, the elements of the initial chromosome vector are initialized with random values. Subsequently, the element values of an initial chromosome vector are updated by referring to the related profile field.

For example, when the profile type is MEM, its corresponding element value of an initial chromosome vector is changed to match an element value corresponding to the related profile.

As illustrated in FIG. 3, the profile “5964F1CB,” with a profile type of MEM, is related to the profile “3FE033B5.” Therefore, the element value corresponding to the profile “5964F1CB” in the initial chromosome vector is changed to match the element value corresponding to the profile “3FE033B5.”

FIG. 5 illustrates an initial chromosome vector, generated under the assumption that there are 8 profiles stored in the profile database 120. Therefore, an initial chromosome vector includes 8 elements.

Next, values of the execution location field in FIG. 3 are set using each N-th generation chromosome vector, and a clustering program is executed using a synthetic data set accordingly to derive a score based on a predetermined index at S20.

A predetermined indicator can be selected in relation to performance expected for the data processing system 1. For example, instructions per cycle (IPC), execution time, etc. can be used as predetermined indicators.

FIG. 5 shows four scores calculated corresponding to four N-th generation chromosome vectors X1, X2, X3, and X4.

Afterwards, it is determined whether N is greater than the maximum number of generations at S30. The maximum number of generations is predetermined to be sufficiently large to allow chromosome vectors to converge. Since the convergence conditions of the chromosome vectors can be determined in various ways by applying a conventional genetic algorithm technology, detailed disclosure is omitted.

If N is not greater than the maximum number of generations, parent chromosome vector pairs for generating a plurality of next generation chromosome vectors are determined at S40.

The parent chromosome vector pairs are stochastically selected from the plurality of N-th generation chromosome vectors. At this time, the probability that any one N-th generation chromosome vector is selected is proportional to a corresponding score. At this time, each chromosome vector may be selected repeatedly.

FIG. 5 shows four parent chromosome vectors P1, P2, P3, and P4 selected from the four N-th generation chromosome vectors.

Parent chromosome vectors sequentially form parent chromosome vector pairs and next generation chromosome vectors are generated from parent chromosome vector pairs.

A plurality of (N+1)-th generation chromosome vectors are generated from the parent chromosome vector pairs, and in this process, crossover and mutation techniques based on genetic algorithms can be applied at S50. Since the crossover and mutation techniques themselves are conventional techniques, descriptions thereof will be omitted.

FIG. 5 shows two (N+1)-th generation chromosome vectors Y1 and Y2 generated from a first parent chromosome vector pair (P1, P2) and two (N+1)-th generation chromosome vectors Y3 and Y4 generated from a second parent chromosome vector pair (P3, P4).

Afterwards, the generation variable N is increased by 1 at S60, and the process returns to step S20 and the above-described operations are repeated.

If N is greater than the maximum number of generations, a chromosome vector among the plurality of N-th generation chromosome vectors is selected as an optimal chromosome vector and the execution locations are finally determined based on the optimal chromosome vector at S70. In an embodiment, a chromosome vector corresponding to the highest score among the plurality of N-th generation chromosome vectors is selected as the optimal chromosome vector.

FIG. 5 shows that the execution location corresponding to each profile is determined by the host 10 or the remote processing device 20 according to the optimal chromosome vector.

When the above-described profile operation and optimization operation are completed, the original dataset and the clustering algorithm are applied to the data processing system 1 to perform a clustering operation.

Although various embodiments have been illustrated and described, various changes and modifications may be made to the described embodiments without departing from the spirit and scope of the invention as defined by the following claims.

What is claimed is:

1. A data processing system, comprising:
a host configured to execute a program for processing a given dataset; and
a remote processing device coupled to the host via an interface,
wherein the host includes:
a profile control circuit configured to generate a profile corresponding to a function that is called during execution of the program;
a profile database storing execution location of the function corresponding to the profile; and
a policy execution circuit configured to allocate the function called during the execution of the program to the host or the remote processing device based on the execution location stored in the profile database.

2. The data processing system of claim 1, wherein the profile control circuit generates the profile using an array of return addresses of functions stored in a stack memory.

3. The data processing system of claim 1, further comprising a policy decision circuit configured to perform an optimization operation to decide an optimal execution location corresponding to the profile that is stored in the profile database, while the program is executed with a synthesized dataset having a size smaller than that of the given dataset,
wherein the optimization operation is performed after performing a profile operation to store, in the profile database, profiles corresponding to functions, which are called, while the program is executed.

4. The data processing system of claim 3, wherein the policy decision circuit performs the optimization operation using a genetic algorithm, and generates an initial chromosome vector during the optimization operation, the initial chromosome vector including a plurality of elements that correspond to a plurality of profiles stored in the profile database.

5. The data processing system of claim 4, wherein the policy decision circuit randomly sets each of the plurality of elements of the initial chromosome vector to a value corresponding to the host or the remote processing device.

6. The data processing system of claim 5, wherein when a first element of the initial chromosome vector corresponds to a profile corresponding to a memory operation, the policy decision circuit sets a value of a second element to match a value of the first element when the second element corresponds to a profile corresponding to a computation operation that is related with the memory operation.

7. The data processing system of claim 5, wherein the policy decision circuit determines parent chromosome vector pairs from a plurality of N-th generation chromosome vectors, and generates a plurality of (N+1)-th generation chromosome vectors from the parent chromosome vector pairs, and wherein N is a non-negative integer and the initial chromosome vector corresponds to a 0-th generation chromosome vector.

8. The data processing system of claim 7, wherein the policy decision circuit determines an optimal chromosome vector among the plurality of N-th generation chromosome vectors and stores the optimal chromosome vector in the profile database when the N is greater than a maximum number of generations.

9. An operation method of a data processing system including a host executing a program that processes a given dataset and a remote processing device coupled to the host via an interface, the operation method comprising:
performing a profile operation to store a plurality of profiles corresponding to a plurality of functions called during execution of the program, the program being executed with a synthesized data-set having a size smaller than that of the given dataset; and
performing an optimization operation to decide an optimal execution location for each of the plurality of functions, either on the host or the remote processing device, while the program is executed with the synthesized dataset.

10. The operation method of claim 9, wherein performing the profile operation includes generating a profile corresponding to a function, which is called during the execution of the program, based on an array of return addresses of functions stored in a stack memory.

11. The operation method of claim 9, wherein performing the optimization operation includes generating an initial chromosome vector including a plurality of elements that correspond to the plurality of profiles, and randomly sets each of the plurality of elements of the initial chromosome vector to a value corresponding to the host or the remote processing device.

12. The operation method of claim 11, wherein when a first element of the initial chromosome vector corresponds to a profile corresponding to a memory operation, performing the optimization operation further includes setting a value of a second element to match a value of the first element when the second element corresponds to a profile corresponding to a computation operation that is related with the memory operation.

13. The operation method of claim 11, wherein performing the optimization operation further includes:
determining parent chromosome vector pairs from a plurality of N-th generation chromosome vectors; and
generating a plurality of (N+1)-th generation chromosome vectors from the parent chromosome vector pairs,
wherein N is a non-negative integer and the initial chromosome vector corresponds to a 0-th generation chromosome vector.

14. The operation method of claim 13, wherein performing the optimization operation further includes:
determining an optimal chromosome vector among the plurality of N-th generation chromosome vectors when the N is greater than a maximum number of generations.

15. The operation method of claim 14, further comprising:
deciding execution locations of the plurality of profiles based on the optimal chromosome vector; and
allocating a function, which is called during the execution of the program, to either the host or the remote processing device according to the execution locations.
